(19) United States
(12) Plant Patent Application Publication
(10) Pub. No.: US 2018/0168086 P1
(43) Pub. Date: Jun. 14, 2018

(54) Festuca grass named 'Freedom Fire Francy'

(50) ;

(71) Applicants: The United States of America, as Represented by the Secretary of Agriculture, Washington, DC (US); Biograss Sod Farms, Inc., Sandy, UT (US)

(72) Inventors: Jack E. Staub, Logan, UT (US); Matthew Robbins, Hyrum, UT (US); Warren Bell, Millcreek, UT (US)

(21) Appl. No.: 15/530,172

(22) Filed: Dec. 9, 2016

(51) Int. Cl. A01H 5/12

(52) U.S. Cl. PLT/384

(57) ABSTRACT

A new and distinct variety of ornamental grass, denominated ‘Francy’, is a non-spreading, bunch type ornamental grass with a tall stature, multi-colored culms and panicles and is fine-leaved.

Latin name of the genus and species of the plant claimed: ‘FREEDOM FIRE FRANCY’ is a new ornamental grass that is a Festuca idahoensis (4×) X F. ovina (2×) triploid (3×) hybrid.

Variety denomination: The new ornamental grass claimed is of the variety denominated ‘Freedom Fire Francy’, Festuca idahoensis (4×) X F. ovina (2×) triploid (3×) hybrid.

### BACKGROUND OF THE INVENTION

The present invention relates to a new and distinct ornamental grass cultivar designated ‘Freedom Fire Francy’ and botanically known as a sterile naturalized Festuca species triploid hybrid (3×) between F. idahoensis (4×) and F. ovina (2×) (Staub et al., 2014). The source population for ‘Francy’ was collected in 1982, as seed from a native fine-leaved Festuca population near Busby, Mont. on the Charles E. Helvey Ranch (T7S R39E NW1/4Sec 3; 45° 31′39″N 106° 58′25″W) east of the Rosebud River in Big Horn County, Mont., which designated it FEID 9025897. In 2009, a visual inspection of 270 FEID 9025897 cloned plants examined under replication in a Logan UT field nursery indicated that the vast majority (>98%) was sterile (degenerated pistils and stamens) (Staub et al., 2014). Of these plants, 19 were comparatively vigorous, relatively tall, and possessed multi-colored culms under harsh semi-arid conditions. These 19 plants were cloned in 2013 and their culm and panicle coloration, relative plant vigor, and plant habit was compared under replication to commercial Festuca cultivars over two years (2014 and 2015) at three semi-arid locations in Logan, Kaysville, and Vernon, Utah. Regardless of year and location, experimental ARS FEID-33 (‘Francy’) was one of four clones that received the highest color ratings (intensity and breath of hues) among the experimental clones and controls examined. Although the culm and panicle color intensity may vary depending on growing conditions, the plant habit and culm and panicle hues ‘Francy’ have been determined to be stable over successive generations of cloning. Culms and panicles of ‘Francy’ exhibit primarily intense pink, salmon, purple, orange, yellow, gold, and maroon coloration depending on growing location.

### BRIEF DESCRIPTION OF THE PHOTOGRAPHS

FIG. 1 is a color photograph showing coloration of a three-year old Festuca ornamental grass ‘Francy’ as nearly true as it is reasonably possible to make in a color illustration of this type.

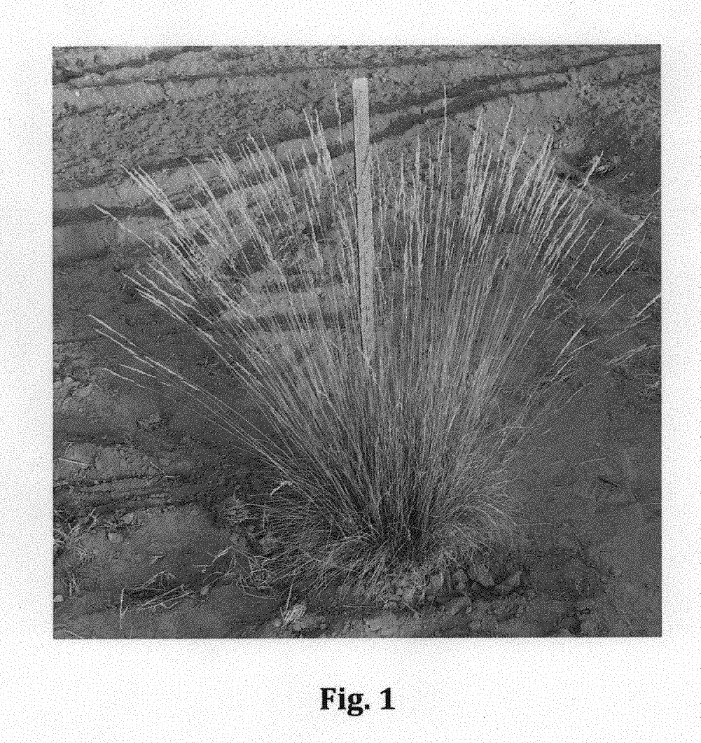

### DETAILED DESCRIPTION OF THE NEW CULTIVAR

The following description of ‘Freedom Fire Francy’ is based on observations taken over two years (2014-2015) of cloned plants under replication (4) in three semi-arid western U.S. growing locations after an initial establishment year. Color data (except those in common terms) are described from Royal Horticulture Society (R.H.S.) color chart designations (Edition V; London, England).

The following traits have been repeatedly observed and represent the characteristics of ‘Francy’ under three ecologically diverse environments in the Great Basin of the western United States [Logan (41° 45′56.01″N 111° 48′39.69″W), Kaysville (41° 1′17.36″N 111° 56′17.95″W), and Vernon (40° 7′19.18″N 112° 26′12.20″W), Utah]. These reproductive and color (culm and panicle) attributes in combination distinguish ‘Francy’ from all other Festuca rangeland cultivars and FEID 9025897 selections.
1. ‘Francy’ is fully sterile by examination of floral parts and seed germination ability, and is defined as a hybrid as determined by DNA analysis and flow cytometry (Staub et al., 2014).
2. The culm and panicle coloration (hue) of ‘Francy’ is more diverse (wider range) and intense than standard rangeland native Festuca species [i.e., cultivars Nezpur (4×; F. idahoensis), Joseph (4×; F. idahoensis), Bighorn (2×; F. ovina), Covar (2×; F. valesiaca), and Durar (6×; F. trachyphylla)].
   ‘Francy’ is substantially larger, that is approximately 23 (2014)-30% (2015) based on plant height and more persistent, that is approximately 10 (2014)-11% (2015) based on plant presence, than standard native rangeland Festuca cultivars under non-irrigated western U.S. semiarid growing environments (1,340-1697 m elevation, average 20-year precipitation 287-437 mm) without supplemental irrigation.

The general observations and descriptions are the result of measurements taken on 2 and 3-year old plants under replication in field nurseries in Logan, Kaysville, and Vernon, Utah. Six plants of each experimental clonal selection (designated as a plot) were grown in each of four replications at each location in a randomized complete block design. Plants spaced 0.5 m with rows and 1 m between rows (˜22,000 plants/ha) where vigor in May, culm and panicle coloration in June/July, and size in August vary depending on year and growing location.

The height and width (centimeters) measurements were obtained during the last two weeks of June of each of two years. Plant vigor (“spring green-up”) was assessed by visual ratings of relative size, color intensity, and transition from winter to spring growth during the first two weeks of May. For biomass estimation (dry weight as grams·plant⁻¹), leaves and inflorescence spikes were harvested each year ≈10 cm above ground when inflorescences were dry, and then oven dried at 60° C. Persistence was determined by counting the number of plants alive within each plot at the time of harvest.

Color of the tussock, culm, and panicle were obtained through visual evaluation as assessed once at each location by 4-8 judges in late June or early July when optimal coloration was observed among cloned plants, which was location and year dependent. Judges examined and characterized coloration at the base (tussock), lower third, middle third, and top third of inflorescences of all plants within a plot between 0800 and 1100 HR in each rating area of all entries. Colors assigned by judges were based on alignments with Royal Horticulture Society (R.H.S.) color chart designations (edition V). Judges identified green (RHS 140A), blue-green (RHS 125A), grey (RHS N187D), brown (RHS 165A), yellow (RHS 4A), gold (RHS 7C), orange (RHS N25C), pink (RHS 67D), salmon (RHS 58C), maroon (RHS N79C), and purple (RHS N78A).

The average percentage color distribution for each rating area was calculated over replications and judges for each entry as the sum of the ratings for that color divided by the total number of ratings multiplied by 100 [e.g., % green at the base =(green base ratings/total base ratings)*100]. Additionally, judges assigned an overall color intensity rating of 1 to 5 (0.5 units; 1=no color, 3=moderate color intensity, and 5=vibrant color intensity) to entries by plot (one value per plot), which was then averaged over replications to provide an average relative color (ARC) intensity for each entry.

Where dimensions, sizes, colors and other characteristics are given, it is to be understood such that over multiple years and growing locations the tussock coloration of ‘Francy’ is predominantly green (RHS 125A; 80-88%) with lesser contributions of blue-green (RHS 140A; 0-13%), while that of other high performance Freedom Fire experimental clones ARS FEID-23 (‘Vida’), ARS FEID-251 (‘Heidi’), and ARS FEID-258 (‘Kim’) are predominantly green (RHS 125A; 44-65%) with contributions of blue-green (RHS 140A; 27-53%) coloration depending on year and growing location. Comparatively less brown (RHS 165A) and grey (RHS N187D) coloration was apparent in ‘Francy’ (8 and 0%, respectively) and commercial controls (4 and 0.03%, respectively). Culms and panicles of ‘Francy’ exhibit a broad array of intense orange (RHS N25C; 0-15%), yellow (RHS 4A; 1-8%), gold (RHS 7C; 5-11%), pink (RHS 67D; 10-20%), purple (RHS N78A; 0-20%), salmon (RHS 58C; 1-13%), and maroon (RHS N79C; 24-36%) coloration depending on year and growing location. These values differ appreciably from commercial controls for orange (RHS N25C; 0-1%), yellow (RHS 4A; 7-38%), gold (RHS 7C; 10-32%), pink (RHS 67D; 0-19%), purple (RHS N78A; 0-2%), salmon (RHS 58C; 8-68%), and maroon (RHS N79C; 0-10%) coloration. Characteristics (color and % range) are approximations of averages set forth as accurately as practicable.
General description:
   Plant type.—Semi-evergreen perennial grass.
   Plant habit.—Tall statured grass with tight tussock (base); densely cespitose.
   Hardiness.—U.S.D.A. 5b-8a.
   Culture.—Tolerant to heat, humidity, and drought.
   Disease and pests.—No susceptible or resistance to pests and diseases has been observed.
   Root description.—Fibrous, without rhizomes.
   Relative vigor.—Relatively high early spring vigor (greening: April-May).
Growth and propagation:
   Propagation.—Division.
   Root development from division.—Fully developed to plant about 9 cm wide and 14 cm tall in 9-cm container under controlled environment (greenhouse) conditions.
   Growth rate.—Moderate to high when compared to commercial controls.
   Branching.—Main shoots grown from the base, tillers but no lateral branches.
Foliage description:
   Leaf shape.—Filiform.
   Leaf division.—Simple.
   Leaf base.—Sheathed.
   Leaf apex.—Long and tip can curve downward at tip depending on growing environment.
   Leaf aspect.—Leaves initially emerge upright, then cascade.
   Leaf color.—Green (RHS 140A) to bluegreen (RHS 125A).
   Leaf venation.—Parallel, color same as leaf blade.
   Leaf margins.—Entire.
   Leaf persistence.—Persistent between April-November.
   Leaf diameter.—0.5-1.5 mm.
   Leaf blade.—Conduplicate, abaxil surfaces smooth and adaxial surfaces often scabrous.
   Leaf durability to stress.—High, where no wilting is exhibited when grown under semi-arid conditions of the Great Basin at approximately 1,407 m elevation, average July temperature and annual 20-year precipitation=approximately 31.7° C. and approximately 437 mm, respectively.
Plant size and habit description (values given averaged over all locations and years):
   Plant size (biomass).—Biomass of ‘Francy’ as estimated by dry weight averaged 73 g and ranged between 39-114 g depending on growing location. The mean of commercial rangeland Festuca cultivars examined was 34 g and biomass ranged between 6-72 g.
   Plant culm height.—The plant height of ‘Francy’ averaged 79 cm and ranged between 63-94 cm. The mean of the commercial rangeland Festuca cultivars examined was 64 cm and ranged between 28-80 cm depending on location.
   Plant width.—The plant width of ‘Francy’ averaged 22 cm and ranged between 18-32 cm. The mean of the commercial rangeland Festuca cultivars examined was 21 cm and ranged between 11-31 cm depending on location.
   Plant habit.—Erect.
   Branches.—Usually initially erect and then somewhat procumbent (prostrate) depending panicle size and weight at maturity.
Flower description:
   Flowers.—Florets often visible, but stamens and/or pistils not prominent and sterile. Inflorescences 5-15 cm, loosely contracted or open, with 1-2 branches per node. Panicle coloration light tan (RHS NN155C) to light brown (RHS 156A).
Culm description:
   Surface.—Usually smooth with no projections or ridges, somewhat glabrous (hairs rare), and occasionally scabrous below the inflorescences.
   Color distribution.—Coloration apparent the entire length of culm.
   Average percentage color distribution.—‘Francy’ exhibits substantially high percentages of orange (RHS N25C; 0-15), yellow (RHS 4A; 1-8), gold (RHS 7C; 5-11), pink (RHS 67D; 10-20), purple (RHS N78A; 0-20), salmon (RHS 58C; 1-13), and maroon (RHS N79C; 24-36) compared to the commercial rangeland Festuca cultivars [orange (RHS N25C; 0-1), yellow (RHS 4A; 7-38), gold (RHS 7C; 10-32), pink (RHS 67D; 0-19), purple (RHS N78A; 0-2), salmon (RHS 58C; 8-68), and maroon (RHS N79C; 0-10)] examined depending on growing location. Average relative color intensity (ARC). The ARC of ‘Francy’ averaged 4.3 and ranged between 4.1-4.4. The mean ARC of the commercial rangeland Festuca cultivars examined was 2.9 and ranged between 2.7-3.1 depending on location.

We claim:

1. A new and distinct ornamental Festuca plant named ‘Freedom Fire Francy’, as illustrated and described herein.
